(19) United States
(12) Patent Application Publication
(10) Pub. No.: US 2014/0162970 A1
(43) Pub. Date: Jun. 12, 2014

(54) Treating Cardiac Arrhythmias, Heart Failure, Peripheral Artery Disease and Stroke With Cyclopentyl-Triazolo-Pyrimidine or Derivative Thereof

(71) Applicant: HearlDrug Research, L.L.C., West Friendship, MD (US)

(72) Inventor: Victor L. Serebruany, West Friendship, MD (US)

(73) Assignee: HearlDrug Research, L.L.C., West Friendship, MD (US)

(21) Appl. No.: 13/961,176

(22) Filed: Aug. 7, 2013

Related U.S. Application Data
(63) Continuation of application No. 12/908,255, filed on Oct. 20, 2010, now abandoned.
(60) Provisional application No. 61/253,829, filed on Oct. 21, 2009.

(51) Int. Cl. A61K 31/7076; A61K 31/519

(52) U.S. Cl. CPC A61K 31/7076; A61K 31/519
USPC 514/46; 514/261.1

(57) ABSTRACT

The present invention relates to new methods for treating and/or preventing cardiac arrhythmias, heart failure, stroke and/or peripheral artery disease by administering a composition comprising triazolopyrimidine, or derivative or metabolite thereof. In particular, the present invention relates to methods for treating cardiac arrhythmias, heart failure, stroke and/or peripheral artery disease by administering CycloPentyl-TriazoloPyrimidine.

### RELATED APPLICATION

This application is a continuation of U.S. application Ser. No. 12/908,255, filed Oct. 20, 2010, which claims the benefit of U.S. Provisional Application No. 61/253,829, filed Oct. 21, 2009, entitled “Treating Cardiac Arrhythmias, Heart Failure, Peripheral Artery Disease and Stroke with Cyclopentyl-Triazolo-Pyrimidine or Derivative Thereof” by Victor L. Serebruany.

The entire teachings of the above applications are incorporated herein by reference.

### BACKGROUND OF THE INVENTION

Cardiac arrhythmia, heart failure, peripheral artery disease and stroke are common diagnoses of hospital patients in the United States. Current treatments involve statins, hypotensives, antiarrythmics, diuretics and other therapies that do treat these diseases; however, current treatments can often be improved to achieve better outcomes. Physicians continue to search for better preventive and/or curative treatments for cardiac arrhythmias, heart failure, stroke, and peripheral artery disease.

Hence, a need exists for new treatment options for individuals who have a cardiac arrhythmia, heart failure, peripheral artery disease, and stroke. A further need exists for effective and improved treatments for these individuals.

### SUMMARY OF THE INVENTION

The present invention relates to methods of treating cardiac arrhythmias, heart failure, stroke or peripheral artery disease in an individual. The steps of the method include selecting an individual having cardiac arrhythmias, heart failure, stroke, peripheral artery disease or combination thereof; and administering an effective amount of a cyclopentyl-triazolo-pyrimidine (CPTP) or derivative thereof to the individual. One or more symptoms, markers or test results associated with cardiac arrhythmias, heart failure, or stroke is improved, as compared to the same prior to administration. In an embodiment, CPTP or derivative thereof includes the following composition:

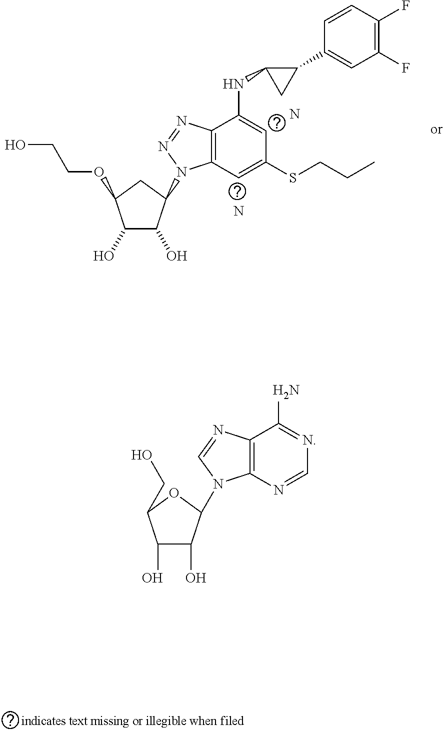

In particular, the methods include treating a cardiac arrhythmia in an individual, wherein the methods include selecting an individual having a cardiac arrhythmia; and administering an effective amount of CPTP or derivative thereof to the individual. After administration of CPTP, the cardiac arrhythmia improves, as compared to the cardiac arrhythmia prior to administration. In an aspect, CPTP is administered orally in an amount between about 5 mg and about 250 mg per day.

In another embodiment, the methods of the present invention pertain to methods of treating an individual with heart failure. The steps of the method embody selecting an individual having an ejection fraction, a brain natriuretic peptide (BNP) level, or combination thereof associated with heart failure; and administering an effective amount of CPTP or derivative thereof to the individual. After administration, the ejection fraction, BNP level, or combination thereof is improved, e.g., by at least about 5%. In an embodiment, the ejection fraction, BNP level, or combination thereof are improved by at least about 10%, 15%, 20%, 25%, 30%, 35%, 40%, 50%, 60%, 70%, 80%, or 90%.

Additionally, the present invention encompasses methods for treating an individual having a stroke. The steps of the method include selecting an individual having an imaging result, wherein the imaging result is indicative of an individual undergoing a stroke. The steps further include administering an effective amount of CPTP or derivative thereof to the individual, as described herein. According to the method, the imaging result, or combination thereof improves, as compared to the same prior to administration, as described herein. Similarly, imaging result improves by at least about 5%, (e.g., by at least about 10%, 15%, 20%, 25%, 30%, 35%, 40%, 50%, 60%, 70%, 80%, or 90%), as compared to the levels prior to the administration.

The present invention encompasses methods for treating an individual having peripheral artery disease (PAD). The steps of the method include selecting an individual having an ankle-brachial index (ABI) between about 1% to about 50%, wherein the index is indicative of an individual undergoing PAD. The steps further include administering an effective amount of CPTP or derivative thereof to the individual, as described herein. According to the method, the ABI improves (e.g., increases), as compared to the same prior to administration, as described herein. In an embodiment, the ABI increases by at least about 5%, (e.g., by at least about 10%, 15%, 20%, 25%, 30%, 35%, 40%, 50%, 60%, 70%, 80%, or 90%), as compared to the levels prior to the administration.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic showing the mechanism of action of CPTP (e.g., Ticagrelor).

### DETAILED DESCRIPTION OF THE INVENTION

A description of preferred embodiments of the invention follows.

The present invention relates to treating a cardiac arrhythmia, heart failure, peripheral artery disease (PAD) and/or stroke by administering CycloPentyl-TriazoloPyrimidine (CPTP) or derivative thereof. Administration of CPTP surprisingly allows for a reduction in symptoms associated with cardiac arrhythmia, heart failure, PAD or stroke. In another embodiment, CPTP administration in these individuals reduces the mortality rate, as compared to individual that do not undergo CPTP administration.

The present invention pertains to methods for preventing or treating an individual at risk for a (e.g., one or more) cardiac arrhythmia, heart failure, PAD and/or stroke. The data described herein show that administering CPTP surprisingly reduces the severity of or prevents these conditions. Prevention of a cardiac arrhythmia, heart failure, PAD and/or stroke (e.g., generally referred to herein as a condition, disorder or disease) refers to delaying or suppressing the onset of the condition, improving test results or markers indicative of the condition, or reducing one or more of its symptoms. To treat an individual at risk for one of these conditions means to alleviate, ameliorate or reduce the severity of one or more of its symptoms.

An individual at risk for a cardiac arrhythmia, heart failure, PAD or stroke refers to an individual with a history of the condition, an individual experiencing at least one symptom of the disorder, an individual having known risk factors (e.g., gender, weight) associated with or caused by the condition, or an individual who has tested positive for the condition using a diagnostic test (e.g., an electrocardiogram, imaging techniques, laboratory tests and the like).

As described above, an embodiment of the invention includes treating individuals who are at risk for cardiac arrhythmia, heart failure, PAD or stroke because they manifest at least one symptom indicative of the condition. A cardiac arrhythmia refers to a condition in which the electrical activity of the heart is not a normal sinus rhythm. Cardiac arrhythmias include, for example, atrial arrhythmias (e.g., Premature Atrial Contractions (PACs), Wandering Atrial Pacemaker, Multifocal atrial tachycardia, Atrial flutter, and Atrial fibrillation (Afib)), junctional arrhythmias (e.g., Supraventricular tachycardia (SVT), AV nodal reentrant tachycardia, Junctional tachycardia, and premature junctional complex), atrio-ventricular arrhythmias (e.g., AV reentrant tachycardia, Wolff-Parkinson-White syndrome, and Lown-Ganong-Levine syndrome), ventricular arrhythmias (e.g., Premature Ventricular Contractions (PVC), Accelerated idioventricular rhythm, Monomorphic Ventricular tachycardia, Polymorphic ventricular tachycardia, and Ventricular fibrillation), and Brugada (e.g., Long QT syndrome), Short QT syndrome, catecholaminergic ventricular tachycardia, and Levi-Lenegre disease. To determine the type of cardiac arrhythmia the individual has, an electrocardiogram (ECG) can be administered and the results interpreted by a cardiologist or other qualified person. A holter monitor, in an embodiment, can be worn by an individual to record heart rhythms over a 24-hour period. In addition to having an abnormal ECG, an individual with a cardiac arrhythmia has certain symptoms indicative of the disease. Symptoms that are indicative of a cardiac arrhythmia, for example, include fatigue, shortness of breath, strong pulse in the neck, or a fluttering, racing heartbeat in the chest, chest discomfort, weak, faint, sweatiness, dizziness, or a combination thereof.

Individuals who are at risk for cardiac arrhythmias also include those having a history of a vascular event (e.g. disorder), including Coronary Heart Disease (CHD), stroke, or Transient Ischemic Attacks (TIAs). Administration of a CPTP to an individual having a cardiac arrhythmia, in an embodiment, results in a reduction of one or more symptoms of the condition by at least about 5% (e.g., 10%, 15%, 20%, 25%, 30%, 35%, 40%, 50%, 60%, 70%, 80%, or 90%). Administration of CPTP also improves the cardiac arrhythmias with which the individual has been diagnosed. Improvement of a cardiac arrhythmia refers to a rhythm that gets closer to a normal sinus rhythm, as compared to the cardiac arrhythmia condition experienced prior to administration.

Heart failure refers to a cardiac condition in which the heart cannot supply the blood required by the body. The cardiac volume output can be measured by an ejection fraction. In particular, an ejection fraction (Ef) is the fraction of blood pumped out of a ventricle with each heart beat. When referring to the left heart ventricles, the ejection fraction can be referred to as the left ventricular ejection fraction (LVEF), and when referring to the right ventrical, then it can be referred to as the ventricular ejection fraction (RVEF). The volume of blood within a ventricle immediately before a contraction is known as the end-diastolic volume and the volume of blood left in a ventricle at the end of contraction is end-systolic volume.

The Ef is determined according this formula:

$$E f = \frac{S V}{E D V} = \frac{E D V - E S V}{E D V}$$

Ejection fraction (Ef)
stroke volume (SV)
end-diastolic volume (EDV)
end-systolic volume (EDV)

Typically, the Ef of a healthy individual is between 50% and 70%. An individual undergoing heart failure has an Ef of less than about 45% (e.g., less than about 40%, 35%, 30%, 25%, or 20%). The range of Ef in an individual undergoing heart failure is between about 10% and about 45%.

In addition to assessing an individual's ejection fraction, a person with heart failure also has certain electrolyte levels and markers that are indicative of heart failure. A marker associated with heart failure includes B-type natriuretic peptide (BNP). In groups of symptomatic patients having heart failure, a diagnostic odds ratio of 27 for BNP compares with a sensitivity of 85% and specificity of 84% in detecting heart failure. In an embodiment, individuals having a BNP level of between about 100 and 20,000 pg/mL is indicative of heart failure. Administration of CPTP, as described herein, results in a reduction of the BNP percentage. In one aspect, the reduction of the BNP occurs by at least about 5% (e.g., by about 10%, 15%, 20%, 25%, 30%, 35%, 40%, 50%, 60%, 70%, 80%, or 90%).

Symptoms of heart failure includes, e.g., shortness of breath, coughing, ankle swelling and reduced exercise capacity. Administration of a CPTP to an individual having a heart failure, in an embodiment, results in a reduction (e.g., improvement) of one or more symptoms or marker indicative of the condition by at least about 5% (e.g., 10%, 15%, 20%, 25%, 30%, 35%, 40%, 50%, 60%, 70%, 80%, or 90%). In particular, CPTP administration improves the ejection fraction, according to the invention, by at least about 5% (e.g., 10%, 15%, 20%, 25%, 30%, 35%, 40%, 50%, 60%, 70%, 80%, or 90%), as compared to the level prior to administration. Improving test results and/or marker levels refers to levels or results that get closer to the normal range for that test or marker, as compared to those prior to CPTP administration.

A stroke refers to a cerebral vascular condition in which blood vessels in the brain are comprised. In particular, cerebrovascular disease (e.g., stroke or transient ischemic attack) is often caused by a thrombosis, an embolism, or a hemorrhagic event. Symptoms of stroke include sudden numbness or weakness of the face, arm or leg, especially on one side of the body, sudden confusion, trouble speaking or understanding, trouble seeing in one or both eyes, trouble walking, dizziness, loss of balance or coordination, or severe headache with no known cause. A number of tests can be performed to ascertain whether an individual is experiencing a stroke. The brain can be imaged using a number of techniques, including, e.g., CT or CAT scan, MRIs, ultrasound, and various angiography techniques. Electroencephalograms (EEGs) can also be used to assess brain activity. These tests are interpreted by a radiologist to determine the presence, absence or degree of a cerebrovascular event. These tests can be performed before, at and/or after CPTP administration. Accordingly, methods of the present invention include administration of CPTP to improve the results interpreted by a radiologist from an imaging technique. Improvement of results from an imaging technique refers to an increase of perfusion to brain tissues, as compared to perfusion prior to administration, and most importantly prevention of repeated strokes.

Similarly, certain markers exist that are indicative of the presence, absence or degree of a stroke. Certain markers are elevated when an individual is undergoing a stroke, and others are not within normal ranges. Administration of CPTP to an individual having a cerebrovascular disease or event, in an embodiment, results in a reduction or improvement of one or more symptoms or marker indicative of the condition by at least about 10% (e.g., 15%, 20%, 25%, 30%, 35%, 40%, 50%, 60%, 70%, 80%, or 90%).

Peripheral artery disease is a condition in which there is an obstruction of an artery in the arms or legs. PAD diagnosis begins with a physical examination. A physician or qualified person will assess for weak pulses in the extremity. The ankle-brachial index (ABI) test is also performed. The ABI test compares the blood pressure in an individual's foot to the blood pressure in your arms to determine how well blood is flowing. This inexpensive test takes only a few minutes and can be performed as part of a routine exam. Normally, the ankle pressure is at least about 90 percent of the arm pressure, but with severe narrowing it may be less than about 50 percent. Accordingly, the present invention involves selecting an individual having an ABI of between about 1% and 50%, and administering CPTP, as described herein. Administration of CPTP to an individual having PAD, in an embodiment, results in an improvement (e.g., increase) of the ABI by at least about 10% (e.g., 15%, 20%, 25%, 30%, 35%, 40%, 50%, 60%, 70%, 80%, or 90%).

The present invention also relates to methods for reducing the occurrence or severity of a cardiac arrhythmia, heart failure, PAD, or stroke in a patient who is at risk for such a disorder. Reducing the occurrence of one of these conditions refers to reducing the probability that a patient will develop the disorder, or delaying the onset of the disorder. Reducing the severity of one of these conditions refers to a reduction in the degree of at least one symptom of the disorder or improving one or more test results indicative of the disorder. The present invention embodies methods for preventing the onset of a cardiac arrhythmia, heart failure, PAD or stroke in an individual by administering CPTP. Use of the methods described herein results in a reduction of at least about 5% (e.g., 10%, 15%, 20%, 25%) in the number of recurrent cardiac arrhythmia, heart failure, PAD and/or stroke.

In one aspect, the present invention involves assessing one or more markers indicative of the condition (e.g., cardiac arrhythmia, heart failure, PAD, or stroke) to determine if the level of one or more markers are elevated, as compared to the level of a mark in an individual without the condition. Individuals with elevated levels of one or more markers are at risk for the condition to which it correlates, as described herein. In an embodiment, administration of CPTP reduces on or more of these markers, as compared to level(s) prior to administration of the CPTP. Controls and assessment of various markers are known in the art. The present invention includes markers that are discovered in the future that are indicative of a cardiac arrhythmia, heart failure, PAD or stroke. CPTP administration prevents, reduces or treats cardiac arrhythmia, heart failure, PAD, or stroke.

One aspect of the invention, as described herein, includes administering a CPTP along with at least one other compound or composition that is used for treating cardiac arrhythmia, heart failure, PAD, or stroke. For an individual with one of these conditions, the CPTP can be administered together with aspirin, heparin, an ADP inhibitor or antagonist (e.g., thienopyridine, such as ticlopidine hydrochloride (marketed under the trademark “Ticlid®” from Roche Laboratories) or clopidogrel bisulfate (marketed under the trademark “Plavix®” from Bristol-Myers Squibb and Sanofi), or GPIIb/IIIa inhibitors (marketed under the trademark “ReoPro®” from Centocor, Inc.; “Integrilin” from Millennium Pharmaceutical, Inc.; “Aggrastat” from Gilford Pharmaceutical). Individuals with cerebrovascular diseases, for example, can receive a CPTP together with Ticlid®, Plavix® or aspirin.

A reduction in the level of one or more markers associated with cardiac arrhythmia, heart failure, PAD, or stroke refers to a decrease in or an absence of the marker, as compared to its level prior to administration or as compared to a control, as further described herein. Levels of one or more markers can be improved, decreased or reduced by at least about 5%, (e.g., 10%, 20%, 30%, 40%, 50%, 60%, 70%, 80%, or 90%), as compared to the level just prior to administration. Hence, one can measure the presence, absence or level of one or more markers and compare the result against a control. For example, one can obtain a suitable sample and compare the level of one or more makers from previous time points (e.g., prior to administration of the CPTP or during the onset of the condition). One can also measure one or more markers in an individual prior to the onset of a cardiac arrhythmia, heart failure, PAD, or stroke (e.g., in a resting state or during a check-up), to determine the individual's baseline. Accordingly, administration of CPTP after the onset of the event or condition modifies levels of the markers (e.g., decreases or increases levels of markers, as compared to those levels during the onset of the event, to reduce the level(s) so that they are closer to baseline (e.g., above, at or below baseline, but less than level(s) during the onset of the condition).

The marker levels assessed can also be compared to a standard or control obtained from normal individuals. In one example, marker levels can be assessed in a population of healthy individuals or individuals who have not had a cardiac arrhythmia, heart failure, PAD, or stroke. Such levels are referred to as a “negative control.” Conversely, levels of markers can also be obtained from a pool of individuals who are undergoing a cardiac arrhythmia, heart failure, PAD, or stroke, e.g., a “positive control.” After administration of CPTP, the level of one or more markers decreases; or the level(s) get closer to the level of the negative control, and further from the positive control. Hence, the methods of the present invention include reducing one or more markers associated with a cardiac arrhythmia, heart failure, PAD or stroke with administration of CPTP wherein one or more levels of such markers are reduced or decreased, as compared to those levels during the occurrence of the event, disease or disorder, or immediately prior to the administration of CPTP.

In another embodiment, the present invention relates preventing the onset of a cardiac arrhythmia, heart failure, PAD, or stroke. An effective amount of at least one CPTP can be administered to prevent one or more marker levels from increasing, or lessen marker levels which would otherwise remain elevated without CPTP administration. For example, an individual who is a risk for a cardiac arrhythmia, heart failure, PAD, or stroke can take CPTP on a daily basis (or every other day), to prevent marker levels from increasing as compared to a control or baseline. Baseline levels of the marker levels can be obtained prior to and/or during the course of administration of a CPTP. The marker levels can stay the same, or can even decrease. Similarly, marker levels can be compared to a negative or positive control, wherein upon administration of CPTP, the levels are closer to the negative control, than the positive control. However measured, the marker levels are prevented from increasing, thereby preventing the occurrence of a cardiac arrhythmia, heart failure, PAD, or stroke.

The present invention relates to methods of reducing or preventing cardiac arrhythmia, heart failure, PAD, or stroke by administering to an individual an effective amount of a CPTP. The term, “CPTP” refers to a (e.g., one or more) compound, derivative or metabolite thereof that includes Triazolo Pyrimidine, and in particular, a CycloPentyl-TriazoloPyrimidine. Triazolopyrimidines and/or CPTP are described in, for example, U.S. Pat. Nos. 5,756,509; 5,593,996; 6,140,325; and 7,601,833.

Examples of CPTP are Ticagrelor (e.g., AZD6140, marketed under Brillinta® by AstraZeneca), and metabolites thereof. AZD6140 is also known as (1S,2S,3R,5 S)-3-[7-[(1R,2S)-2-(3,4-Difluorophenyl)cyclopropylamino]-5-(propylthio)-3H-[1,2,3]triazolo[4,5-d]pyrimidin-3-yl]-5-(2-hydroxyethoxy)cyclopentane-1,2-diol. In an embodiment, AZD6140 is represented by formula I:

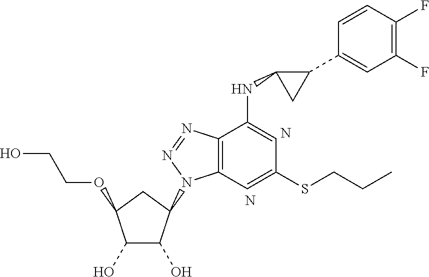

and its metabolite, adenosine, is represented by formula II:

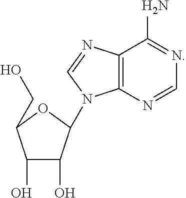 [SMILES: Nc1ncnc2c1ncn2C1OC(CO)C(O)C1O]

The present invention encompasses CPTP that are currently used, or those later discovered or formulated. CPTP includes biologically active portions, groups or fragments thereof. Examples of Triazolopyrimidines that can be used in the administration of the present invention further include Tradipi (phosphodiesterase inhibitor and as a competitive inhibitor of the platelet-derived growth factor (PDGF) receptor restenosis)1, essramycin (antibiotic), NK026680 (transplant graft rejection), 5-chloro-6-(trifluorophenyl)-N-fluoroalkyl[1,2,4]triazolo[1,5-a]pyrimidin-7-amine (anticancer), oxazolopyridines (dual thrombin/Factor Xa inhibitors, anticoagulants); 2-furanyl[1,2,4]triazolo[1,5-a][1,3,5]triazine—adenosine A2 receptor antagonists for Parkinson Disease; fused 1,2,4-triazolo[1,5-c]pyrimidine derivatives as human adenosine A3 receptor ligands.

Methods of making CPTP are known in the art. CPTP can be made synthetically, as described in, for example, in U.S. Pat. Nos. 5,756,509; 5,593,996; 6,140,325; and 7,601,833.

### Modes and Manner of Administration, Dosages

CPTP used in the present invention can be administered with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include potassium sorbate, sodium metabisulfite, methyl paraben, propyl paraben, thimerosal, etc. The compositions can also be combined where desired with other active substances, e.g., enzyme inhibitors, to reduce metabolic degradation. A carrier (e.g., a pharmaceutically acceptable carrier) is preferred, but not necessary to administer the compound.

The CPTP can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

The CPTP used in the invention can be administered intravenously, parenterally, intramuscular, subcutaneously, orally, nasally, topically, by inhalation, by implant, by injection, or by suppository. The composition can be administered in a single dose or in more than one dose over a period of time to confer the desired effect. In one embodiment, AZD6140, can be administered orally in an amount between about 2 mg and about 250 mg/daily (e.g., about 10, 20, 40, 80, 100, 150, or 200 mg/daily).

The actual effective amounts of a compound or drug can vary according to the specific composition being utilized, the mode of administration and the age, weight and condition of the patient. For example, as used herein, an effective amount of the drug is an amount which reduces the platelet activation state. Dosages for a particular individual patient can be determined by one of ordinary skill in the art using conventional considerations, (e.g. by means of an appropriate, conventional pharmacological protocol).

For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used.

Pulmonary administration can be accomplished, for example, using any of various delivery devices known in the art such as an inhaler. See. e.g., S. P. Newman (1984) in Aerosols and the Lung, Clarke and Davis (eds.), Butterworths, London, England, pp. 197-224; PCT Publication No. WO 92/16192; PCT Publication No. WO 91/08760.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

The administration of CPTP and any additional compound used to treat a cardiac arrhythmia, heart failure, PAD, or stroke can occur simultaneously or sequentially in time. The compound can be administered before, after or at the same time as the CPTP. Thus, the term “co-administration” is used herein to mean that the CPTP and the additional compound will be administered at times to achieve a reduction or treatment of the cardiac arrhythmia, heart failure, PAD, or stroke. The methods of the present invention are not limited to the sequence in which the CPTP and additional compound are administered, so long as CPTP and additional compound are administered close enough in time to produce the desired effect.

Marker levels can be assessed using methods that are currently known in the art, as well as those that are later discovered.

### EXEMPLIFICATION

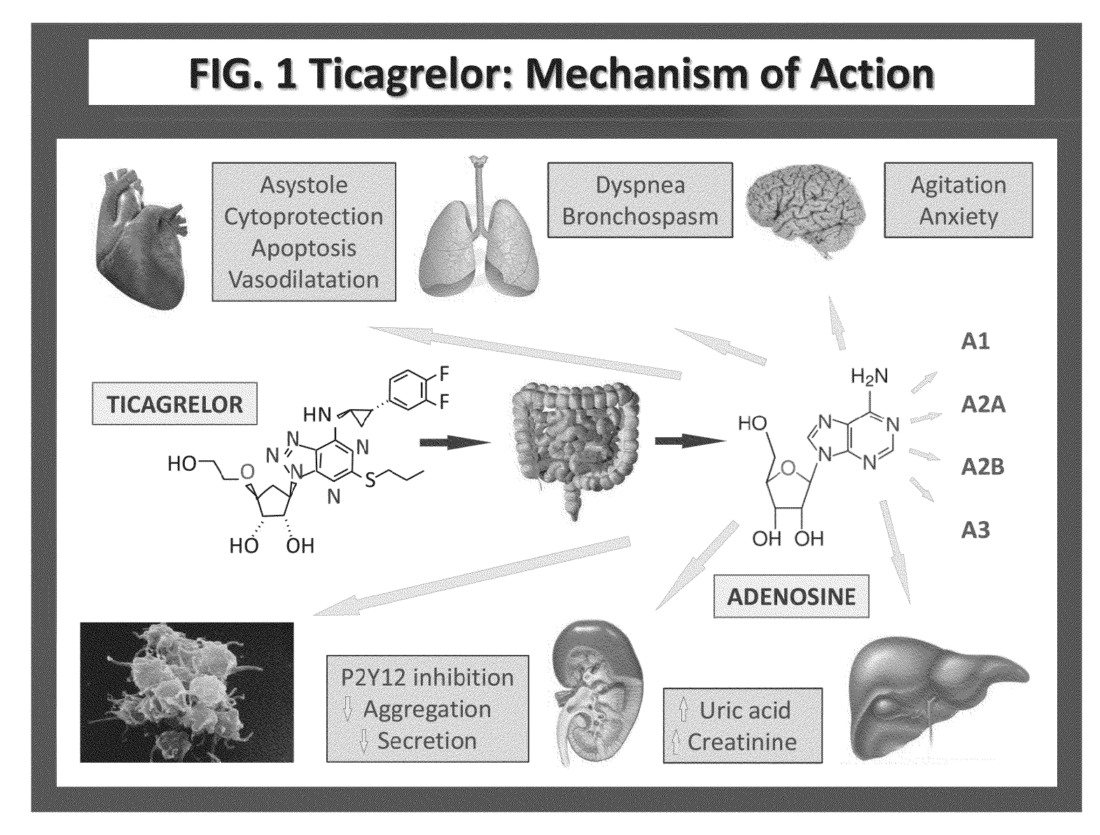

In studies performed, a remarkable outcome was obtained when CPTP was administered to individuals undergoing coronary stent implantation for therapy of Acute Coronary Syndrome (ASC). The primary end point in a large randomized study was the time of the first event of death from vascular causes, myocardial infarction (MI) or stroke, and occurred in 11.7% of patients treated with clopidogrel, versus 9.8% of patients randomized to CPTP, representing a highly significant benefit (HR=0.84; CI=0.77-0.92; p<0.001) of the cyclopentyl-triazolo-pyrimidine. Triaging these three components of the combined end point suggests that the difference in favor of CPTP was driven by the reduction of vascular death (p<0.001), and MI (p<0.005), but not stroke (p=0.22). The mechanistic cornerstone of the outcome after ticagrelor is most likely directly related to the up-regulation of the adenosine receptors. In addition to causing reversible platelet inhibition, adenosine is involved in numerous biological activities including cardioprotection from reperfusion injury, apoptosis, myocyte regeneration, improved myocardial contractility, and electrical stability. Although ticagrelor is not an ATP analogue, and may not cause massive adenosine overload, changes in the adenosine metabolism are critical for the comprehension of the PLATO results. Potential mechanisms that target the adenosine metabolism through ticagrelor and which may affect vascular outcomes, platelets, and associated side effects, are presented in FIG. 1.

Beyond Platelets: Based on the analyses of recent trials, the vascular outcome benefit of ticagrelor cannot be explained by faster and more potent platelet inhibition alone when compared with clopidogrel. The lack of a long-term advantage and of a mortality benefit in a trial, as well as identical death rates in another study, clearly underscore the idea that low platelet responsiveness after clopidogrel, the so-called “resistance” cause worsened vascular outcomes. While the antiplatelet potency of the ticagrelor dose used in one study matched closely with the prasugrel dosing regimen used in another study, the magnitude and timing of outcome patterns are entirely different. Therefore, it is likely not the faster speed of action, nor the higher potency of platelet inhibition with ticagrelor compared to clopidogrel, but clearly something beyond pure P2Y12 receptor inhibition by this novel compound, cyclopentyl-triazolo-pyrimidine, now known as ticagrelor. Considering that ticagrelor is a “first-in-class” type of drug, and it is not a thienopyridine like ticlopidine, clopidogrel, and prasugrel, it seems that ADP-receptor blockade may not be the most important commodity of ticagrelor. Most likely the mechanism responsible for such benefit is complex, related to the alterations of chronic adenosine modulation by purinoreceptors in blood, thereby potentially improving myocardial contractility, vascular tone, and directly protecting cardiomyocytes. Considering that the adenosine receptors A1, A2A, A2B, and A3 modulate oppositely directed physiological functions, their interplay and differential upregulation can explain the some proarrhythmic properties of ticagrelor early during drug administration, with prevention of ventricular tachyarrhythmias and sudden death late in the trial. Since ticagrelor prevented more deaths than myocardial infarctions (MI), it is likely that administration of ticagrelor can prevent fatal ventricular tachycardias and sudden death, as well as other arrhythmias, and heart failure benefit rather than thrombotic occlusions alone.

In summary, the study revealed a remarkable advantage of ticagrelor. Unless the regulatory authorities discover serious flaws with the study, which is unlikely, the drug can substantially change the present landscape of oral antiplatelet therapy, especially in high-risk patients. Despite a somewhat unfavorable safety profile, ticagrelor has a lot of room to compensate for these well defined side effects based on a documented absolute mortality reduction, solid prevention of MI, and convincing pattern of growing over time benefit.

Table 1 is a listing of investigator assignments for cause of death. Causes of death were similar between treatment groups. The most common cause of death was myocardial infarction occurring in about 1% of randomized patients. Sudden death, heart failure, other vascular events and stroke were among the more common causes of death.

TABLE 1
Listed Causes of Death from Efficacy Data Set
(by randomized treatment)

| | Randomised Treatment | |
|---|---|---|
| | Ticagrelor | Clopidogrel |
| | 80 mg bd | 75 mg bd |
| Characteristic | N = 9333 | N = 9291 |
| Aortic dissection | 1 (0.0%) | 2 (0.0%) |
| Arterial embolism | 5 (0.0%) | 2 (0.0%) |
| Cancer | 14 (0.2%) | 17 (0.2%) |
| Cardiac arrhythmia | 20 (0.2%) | 28 (0.3%) |
| Death from bleeding (not related to trauma) | 13 (0.1%) | 15 (0.2%) |
| Endocarditis | 0 (0.0%) | 0 (0.0%) |
| Heart failure | 81 (0.5%) | 82 (0.7%) |
| Liver failure | 5 (0.0%) | 1 (0.0%) |
| Multiorgan failure | 9 (0.1%) | 14 (0.2%) |
| Myocardial infarction | 89 (1.0%) | 88 (0.9%) |
| Other coronary artery disease | 8 (0.0%) | 4 (0.0%) |
| Other non-vascular cause | 8 (0.1%) | 11 (0.1%) |
| Other vascular cause | 44 (0.5%) | 55 (0.6%) |
| Pneumonia | 20 (0.1%) | 8 (0.1%) |
| Pulmonary embolism | 2 (0.0%) | 8 (0.1%) |
| Renal failure | 2 (0.0%) | 5 (0.1%) |
| Respiratory failure | 13 (0.1%) | 12 (0.1%) |
| Ruptured Aortic ansurysm | 1 (0.0%) | 0 (0.0%) |
| Sepais | 7 (0.1%) | 23 (0.2%) |
| Stroke | 20 (0.2%) | 18 (0.2%) |
| Sudden death | 80 (0.6%) | 77 (0.8%) |
| Suicide | 1 (0.5%) | 1 (0.0%) |
| Trauma | 3 (0.0%) | 1 (0.0%) |
| Unstable Angina | 7 (0.1%) | 8 (0.1%) |
| Valvular disease | 0 (0.0%) | 1 (0.0%) |
| Vascular death, sub-classification missing | 0 (0.0%) | 1 (0.0%) |
| Unknown | 39 (0.4%) | 58 (0.8%) |

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details can be made therein without departing from the scope of the invention encompassed by the appended claims.

What is claimed is:

1. A method of treating causes of mortality, wherein the causes of mortality consist essentially of heart failure and sudden death in an individual, wherein the method comprises:
a) selecting an individual having an increased risk of heart failure, sudden death, or a combination thereof; and
b) administering an amount of a cyclopentyl-triazolo-pyrimidine (CPTP) or derivative thereof to the individual, wherein the amount is between about 5 mg and about 250 mg per day;
wherein one or more symptoms, markers, or test results associated with heart failure, or sudden death are improved, as compared to the same prior to step b).

2. The method of claim 1, wherein the CPTP or derivative thereof has a composition that comprises:

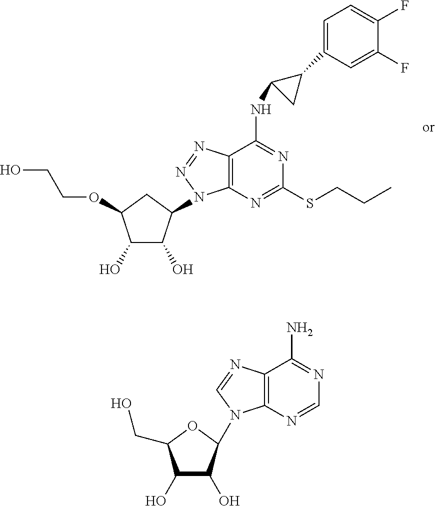

3. The method of claim 1, wherein an ejection fraction, brain natriuretic peptide (BNP) level, or a combination thereof for heart failure is improved, as compared to the same prior to step b).

4. A method for treating an individual having heart failure, wherein the method comprises:
a) selecting an individual having an ejection fraction, a BNP level, or a combination thereof associated with heart failure; and
b) administering an amount of CPTP or derivative thereof to the individual, wherein the amount is between about 5 mg and about 250 mg per day;
wherein the ejection fraction, BNP, or a combination thereof is improved, as compared to the same prior to step b).

5. The method of claim 4, wherein CPTP is administered orally.

6. The method of claim 4, wherein the ejection fraction, BNP, or a combination thereof is improved by at least about 5%, as compared to the levels prior to the administration of step b).

7. A method of reducing a risk of death due to sudden death in an individual, wherein the method comprises:
a) selecting an individual at an increased risk for sudden death; and
b) administering an amount of CPTP or derivative thereof to the individual, wherein the amount is between about 5 mg and about 250 mg per day;
wherein the individual's risk of sudden death decreases as compared to prior to step b).

8. The method of claim 7, wherein CPTP is administered orally.
